(19) United States
(12) Patent Application Publication
(10) Pub. No.: US 2008/0003157 A1
(43) Pub. Date: Jan. 3, 2008

(54) METHODS AND APPARATUS FOR PFC ABATEMENT USING A CDO CHAMBER

(75) Inventors: Sebastien Raoux, Santa Clara, CA (US); Kuo-Chen Lin, Taipei City (TW); Robbert M. Vermeulen, Pleasant Hill, CA (US); Daniel O. Clark, Pleasanton, CA (US); Stephen Tsu, Los Gatos, CA (US); Mehran Moalem, Cupertino, CA (US); Allen Fox, Sunnyvale, CA (US); Monique McIntosh, San Jose, CA (US); Joshua Putz, Fairfield, CA (US); Eric Rieske, Livermore, CA (US); Poh Soh Lee, Singapore (SG)

Correspondence Address: DUGAN & DUGAN, PC, 55 SOUTH BROADWAY, TARRYTOWN, NY, 10591, US

(73) Assignee: APPLIED MATERIALS, INC.

(21) Appl. No.: 11/673,404

(22) Filed: Feb. 9, 2007

Related U.S. Application Data
(60) Provisional application No. 60/772,317, filed on Feb. 11, 2006.
(60) Provisional application No. 60/865,347, filed on Nov. 10, 2006.

(51) Int. Cl. B01D 53/74; C01B 21/00; C01B 7/20; C07C 1/00

(52) U.S. Cl. 423/239.1; 422/173; 423/241; 585/641; 585/702

(57) ABSTRACT

In at least one aspect, a controlled decomposition oxidation (CDO) system is provided for abating perfluorocarbons (PFCs) that includes (1) an upstream portion including a first conduit adapted to convey a gaseous waste stream; (2) a thermal reaction chamber having an inlet coupled to the first conduit, a catalyst bed adapted to abate PFCs, and an outlet; and (3) a downstream portion including a second conduit having a first end coupled to the outlet of the thermal reaction chamber and having a portion, downstream from the first end, positioned proximate to the first conduit. The second conduit is adapted to convey a gaseous waste stream heated within the thermal reaction chamber to enable a transfer of heat energy from the second conduit to the first conduit so as to pre-heat the gaseous waste stream in the first conduit. Numerous other aspects are provided.

The present application claims priority to U.S. Provisional Patent Application Ser. No. 60/772,317, filed Feb. 11, 2006 and entitled “METHODS AND APPARATUS FOR PFC ABATEMENT USING A CDO CHAMBER”, (Attorney Docket No. 10910/L) and U.S. Provisional Patent Application Ser. No. 60/865,347, filed Nov. 10, 2006 entitled “METHODS AND APPARATUS FOR PFC ABATEMENT USING A CDO CHAMBER”, (Attorney Docket No. 10910/L2), each of which is hereby incorporated herein by reference in its entirety for all purposes.

### FIELD OF THE INVENTION

The present invention relates to semiconductor device manufacturing, and more specifically to methods and apparatus for PFC abatement using a CDO chamber.

### BACKGROUND OF THE INVENTION

Many of the processes used during semiconductor device manufacturing, such as metal and dielectric etch processes, produce undesirable by-products including perfluorocompounds (PFCs) or by-products that may decompose to form PFCs. Cleaning processes used to remove materials accumulated on chamber components of deposition chambers, such as chemical or physical vapor deposition chambers, also may produce PFCs. Methods and apparatus for abating such PFCs are desirable.

### SUMMARY OF THE INVENTION

In some aspects, a method is provided for abating perfluorocarbons (PFCs) in a gaseous waste abatement system having a pre-installed controlled decomposition oxidation (CDO) thermal reaction chamber that includes (1) providing a catalyst bed within the CDO thermal reaction chamber; and (2) introducing a gaseous waste stream into the CDO thermal reaction chamber so as to expose the gaseous waste stream to the catalyst bed.

In certain aspects, a system is provided for abating perfluorocarbons (PFCs) from a gaseous waste stream that includes (1) a wet scrubber adapted to scrub a gaseous waste stream and having an outlet adapted to discharge a scrubbed gaseous waste stream; and (2) a controlled decomposition oxidation (CDO) system. The CDO system includes a CDO thermal reaction chamber that includes (a) an inlet coupled to the outlet of the wet scrubber; (b) a catalyst bed adapted to abate PFCs within the CDO thermal reaction chamber; and (c) an outlet.

In some other aspects, a method is provided for abating perfluorocarbons (PFCs) in a gaseous waste abatement system having a controlled decomposition oxidation (CDO) thermal reaction chamber that includes (1) providing a catalyst bed within the CDO thermal reaction chamber; (2) conveying a gaseous waste stream past a heat exchanger into an inlet of the CDO thermal reaction chamber and to the catalyst bed; (3) filtering the gaseous waste stream through the catalyst bed, the filtered gaseous waste stream being heated in the catalyst bed; and (4) recirculating the heated gaseous waste stream from the catalyst bed to the heat exchanger.

In at least one aspect, a controlled decomposition oxidation (CDO) system is provided for abating perfluorocarbons (PFCs) that includes (1) an upstream portion including a first conduit adapted to convey a gaseous waste stream; (2) a thermal reaction chamber having an inlet coupled to the first conduit, a catalyst bed adapted to abate PFCs, and an outlet; and (3) a downstream portion including a second conduit having a first end coupled to the outlet of the thermal reaction chamber and having a portion, downstream from the first end, positioned proximate to the first conduit. The second conduit is adapted to convey a gaseous waste stream heated within the thermal reaction chamber to enable a transfer of heat energy from the second conduit to the first conduit so as to pre-heat the gaseous waste stream in the first conduit.

In some other aspects, a system is provided for abating perfluorocarbons (PFCs) that includes (1) an upstream portion including a first conduit adapted to convey a gaseous waste stream and a heating device coupled to the first conduit and adapted to pre-heat the gaseous waste stream; and (2) a thermal reaction chamber including an inlet coupled to the first conduit and a catalyst bed adapted to abate PFCs in the gaseous waste stream entering the thermal reaction chamber from the first conduit.

In certain other aspects, a system is provided for abating perfluorocarbons (PFCs) within a gaseous waste stream that includes (1) a first conduit adapted to convey the gaseous waste stream and having an outlet; (2) a heat exchanger positioned in the first conduit proximate to the outlet; (3) a thermal reaction chamber including an inlet coupled to the outlet of the first conduit, a catalyst bed having a catalyst material positioned within the thermal reaction chamber adapted to abate PFCs within the gaseous waste stream; and (4) a second conduit having a first end coupled to the catalyst bed and a second end coupled to the heat exchanger.

In yet other aspects, a system is provided for abating perfluorocarbons (PFCs) within a gaseous waste stream that includes (1) a first conduit adapted to convey the gaseous waste stream and having an outlet; (2) a thermal reaction chamber including an inlet coupled to the outlet of the first conduit, a catalyst bed having a catalyst material positioned within the chamber and adapted to abate PFCs within the gaseous waste stream, and an outlet positioned opposite the inlet; and (3) a second conduit having a first end coupled to the catalyst bed and a second end that extends into the first conduit.

In still other aspects, an apparatus is provided for abating perfluorocarbons (PFCs) in a controlled decomposition oxidation (CDO) thermal reaction chamber. The apparatus includes (1) a cartridge insertable into the thermal reaction chamber having gas-permeable first and second ends and including a catalyst material; and (2) thermally-conductive fixtures positioned within the cartridge.

In yet other aspects, an apparatus is provided for abating perfluorocarbons (PFCs) in a controlled decomposition oxidation thermal reaction chamber. The apparatus includes a cartridge insertable into the thermal reaction chamber having gas-permeable first and second ends and including a catalyst material.

In at least another aspect, an apparatus is provided for abating perfluorocarbons (PFCs) in a controlled decomposition oxidation (CDO) thermal reaction chamber that includes an annular catalyst bed embedded in the thermal reaction chamber having an outer porous liner and an inner porous liner, the inner porous liner positioned within a central region of the thermal reaction chamber so as to define an inner plenum. A gaseous waste stream introduced into the thermal reaction chamber may flow through the outer porous liner through the catalyst bed and into the inner plenum.

In additional aspects, an apparatus is provided for abating perfluorocarbons (PFCs) in a gaseous waste stream that includes (1) a controlled decomposition oxidation (CDO) thermal reaction chamber having an inlet adapted to receive the gaseous waste stream; and (2) a catalyst bed including a catalyst material positioned within the CDO thermal reaction chamber so as to expose the gaseous waste stream to the catalyst material. Numerous other aspects are provided.

Other features and aspects of the present invention will become more fully apparent from the following detailed description, the appended claims and the accompanying drawings.

### BRIEF DESCRIPTION OF THE DRAWINGS

FIG. 1A is a schematic diagram of a PFC abatement system according to at least one embodiment of the invention.

FIG. 1B is a schematic diagram of a first alternative embodiment of the PFC abatement system of FIG. 1A provided in accordance with the present invention.

FIG. 1C is a schematic diagram of a second alternative embodiment of the PFC abatement system of FIG. 1A provided in accordance with the present invention.

FIG. 2 is a top schematic view of an exemplary embodiment of the wet scrubber depicted in FIG. 1A.

FIG. 3 is a cross-sectional view of the wet scrubber of FIG. 2.

FIG. 4 is a cross-sectional view of an alternative embodiment of the wet scrubber of FIG. 3.

FIG. 5 is a partial perspective view of a CDO chamber that may be used as a catalyst bed in accordance with the present invention.

FIG. 6 is a top view of an exemplary embodiment of the catalyst cartridge of FIG. 5.

FIGS. 7A and 7B are a top view and a side view, respectively, of an exemplary reduced-pressure-drop catalyst bed provided in accordance with the present invention.

FIG. 8 illustrates a schematic view of a first apparatus for heating a catalyst bed provided in accordance with the present invention.

FIG. 9 illustrates a schematic view of a second apparatus for heating a catalyst bed provided in accordance with the present invention.

FIG. 10 illustrates a schematic view of a third apparatus for heating a catalyst bed provided in accordance with the present invention.

FIG. 11 illustrates a schematic view of a fourth apparatus for heating a catalyst bed provided in accordance with the present invention.

FIG. 12 is a schematic diagram of an exemplary cross heat exchanger that may be used in accordance with the present invention.

### DETAILED DESCRIPTION

The present invention provides methods and apparatus for PFC abatement. In one or more embodiments of the invention, an existing controlled decomposition oxidation (CDO) chamber used to oxidize toxic materials such as acids, acid gases, hydrides, flammable gasses, etc., may be modified and/or retrofitted to abate PFCs. Use of existing, on-site abatement equipment such as a CDO chamber to abate PFCs can result in a significant cost savings when compared to the expense of installing a new, conventional PFC abatement system.

Exemplary processes that may be abated in accordance with the invention include metal and dielectric etch processes, cleaning processes for chemical vapor deposition, physical vapor deposition or other deposition processes, or the like. Exemplary PFCs that may be abated include CF₄, C₂F₆, C₄F₆, C₃F₈, CHF₃, CH₃F, CH₂F₂, SF₆, by-products of NF₃ cleaning, etc. Other processes may be abated, as may other PFCs.

### System Overview

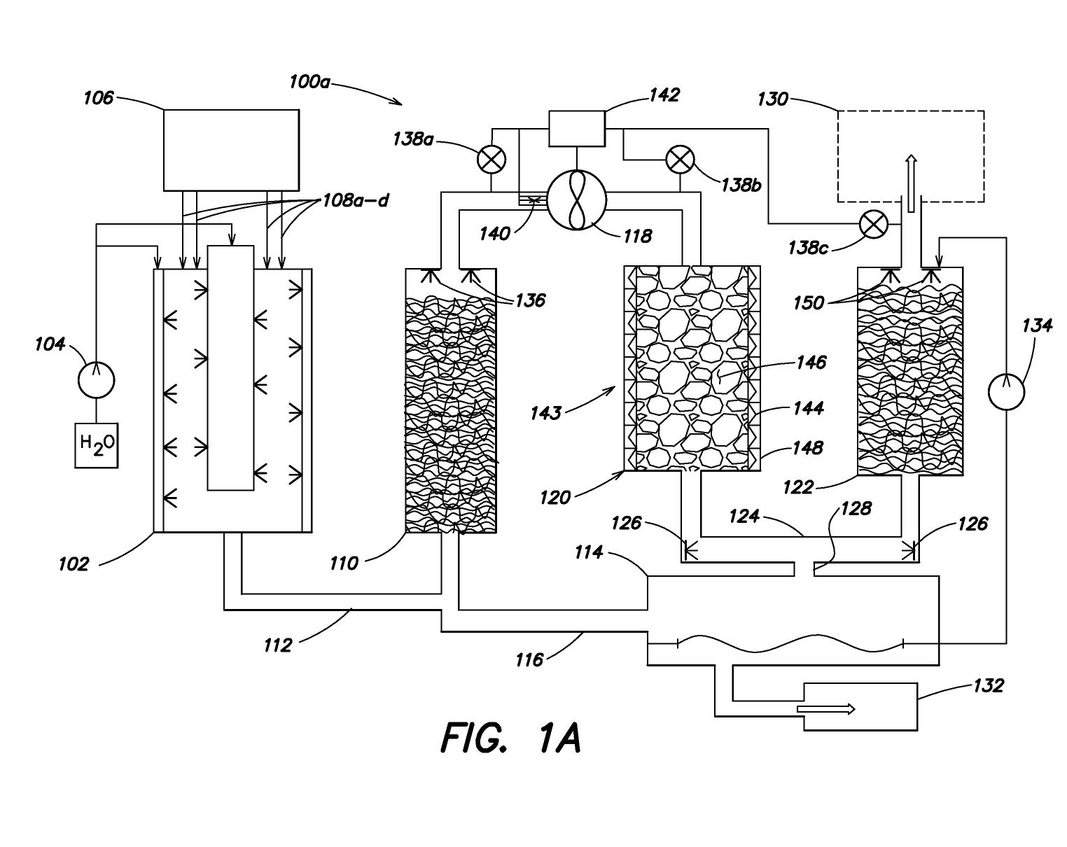

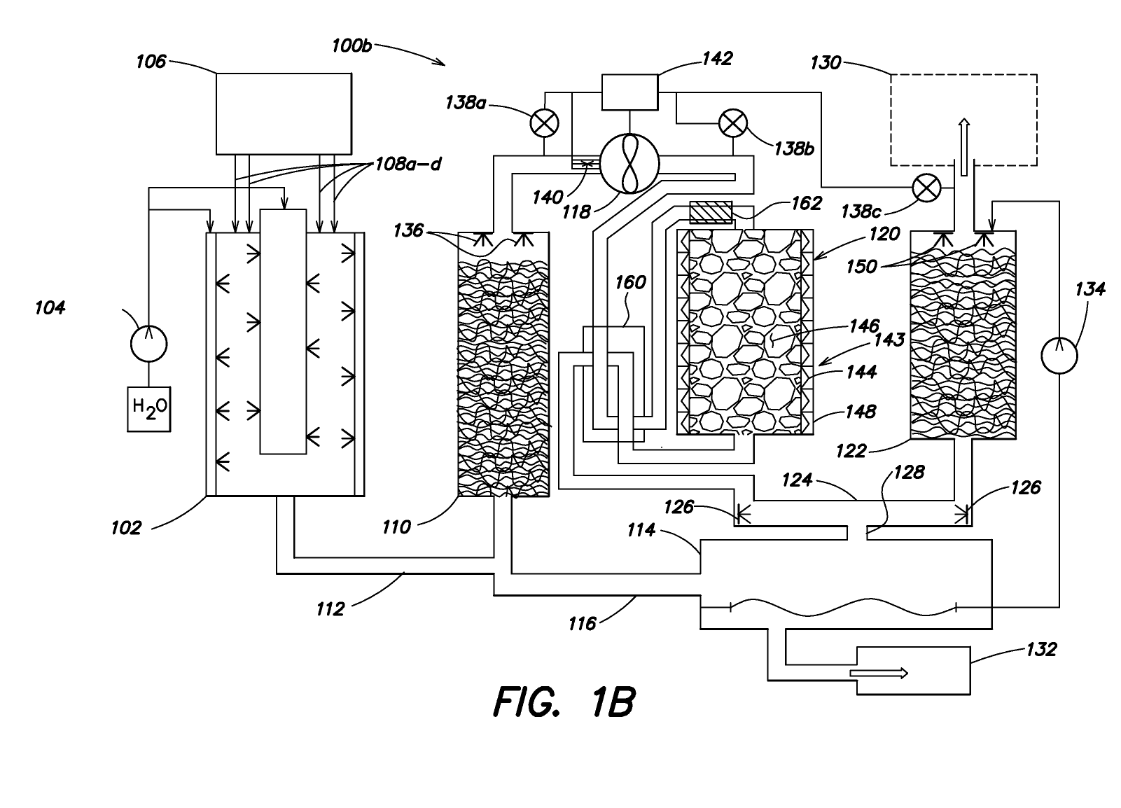

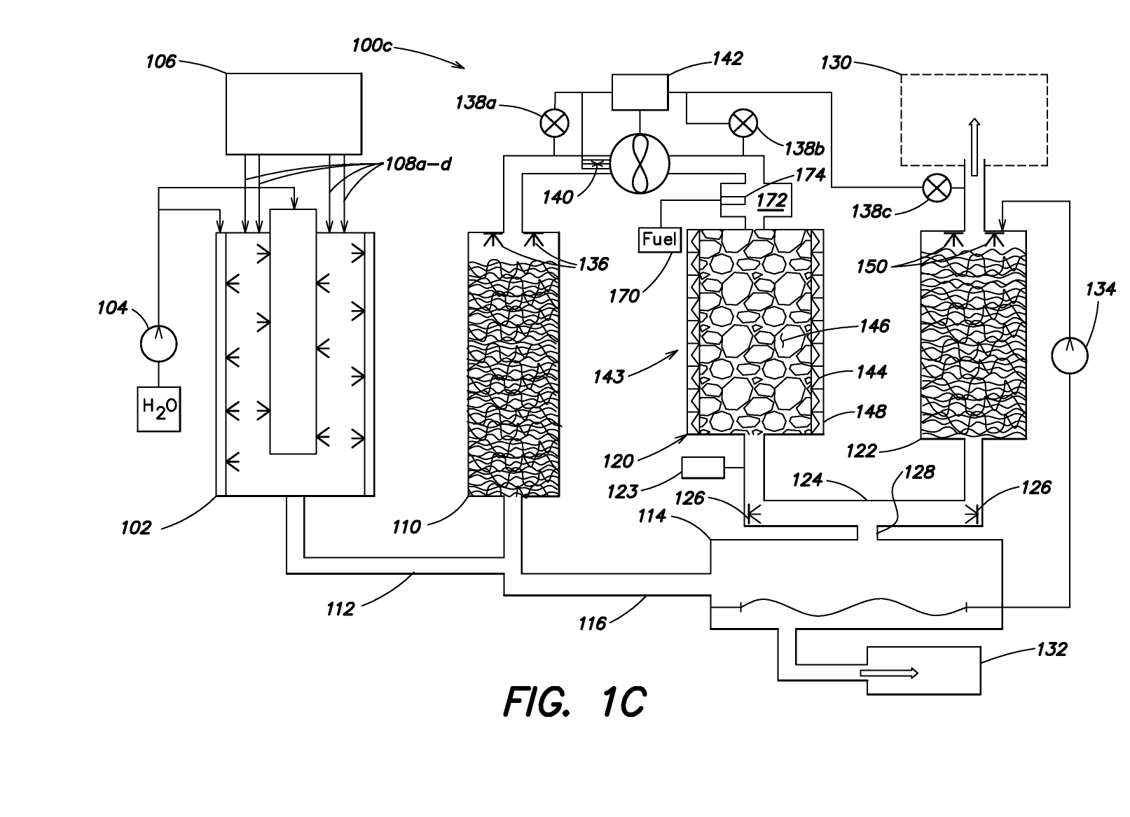

FIG. 1A is a schematic diagram of a first exemplary PFC abatement system 100a according to at least one embodiment of the invention. The abatement system 100a includes a wet scrubber 102, which is fed water (e.g., from house water, a pump, a high pressure pump 104, etc.). Gaseous waste streams from one or more process chambers are directed (e.g., exhausted) into wet scrubber 102. In FIG. 1A, a single process tool 106 is shown that includes four process chambers (as indicated by exhaust lines 108a-d), each being exhausted into the water scrubber 102. It is understood that water scrubber 102 may receive gaseous waste streams from any number of process tools and/or process chambers (e.g., 1, 2, 3, 4, 5, 6, etc.).

Wet scrubber 102 employs a water mist to remove or diminish the presence of one or more contaminants (e.g., SiF₄) from the gaseous waste streams. Preferably, SiF₄ may be reduced to a concentration of approximately less than one part per million. Greater or lesser concentrations of SiF₄ may be achieved.

The processed gaseous waste streams are then directed from wet scrubber 102 to a first packed bed chamber 110 via conduit 112. Contaminants and/or particulates separated from the gaseous waste streams (e.g., HCl, HF, SiO2 suspended in water, etc.) at the wet scrubber 102 may be directed to a sump 114 via a branch or extension 116 of conduit 112. These separated contaminants may be removed by any other appropriate means. Additionally, some amount of the processed gaseous waste stream may be directed to the sump 114 without detriment to the abatement system 100a.

The first packed bed chamber 110 may remove water, contaminants, and/or particulates from the gaseous waste streams. The separated water, contaminants, and/or particulates may be directed to the sump 114 as described above. After passing through the first packed bed chamber 110, the gaseous waste streams may be directed through a blower 118 into a catalyst bed 120. As will be described further below, the catalyst bed 120 interacts with the gaseous waste streams to abate PFCs.

PFC abated gaseous waste streams are directed from the catalyst bed 120 to a second packed bed chamber 122 via conduit 124. While in transit from catalyst bed 120 to second packed bed chamber 122, the abated gaseous waste streams may be cooled by water spray nozzles 126 and/or other means in conduit 124. Water, contaminants, and/or particulates separated from the abated gaseous waste stream in the catalyst bed 120, the conduit 124, and/or the second packed bed chamber 122 are directed to the sump 114 via a branch or extension 128 of conduit 124. After passing through second packed bed chamber 122 the abated gaseous waste streams may be fed to a house exhaust system 130 (shown in phantom) and/or further abatement chambers (not shown).

Water, contaminants, and/or particulates separated from the gaseous waste stream and directed into sump 114 via extensions 116 and 128 may pass, along with any other fluid in sump 114 to an acid waste neutralization system 132. In at least one embodiment, water from the sump 114 may be filtered and recirculated via a recirculation pump 134 to the second packed bed chamber 122 and/or to any other suitable location within the abatement system 100a.

FIG. 1B is a schematic diagram of a first alternative embodiment of the PFC abatement system 100a of FIG. 1A, referred to as PFC abatement system 10b. The PFC abatement system 100b is similar to the PFC abatement system 100a of FIG. 1A, but includes a cross heat exchanger 160 or other recuperator for preheating a gas stream before entry into the catalyst bed 120. Such pre-heating of the gas stream may assist in heating the catalyst used in the catalyst bed 120. The cross heat exchanger 160 employs the gas stream output from the catalyst bed 120, which is heated by the heaters 144 and/or exothermic abatement processes performed within the catalyst bed 120, to pre-heat a gas stream before it enters the catalyst bed 120. Any suitable heat exchanger or recuperator may be used. Exemplary cross heat exchangers are described below with reference to FIGS. 8-12.

Additionally or alternatively, the PFC abatement system 100b may include a pre-heater 162, such as an electric or other suitable heater, for pre-heating a gas stream before it enters the catalyst bed 120. If both a heat exchanger and a pre-heater are employed, a smaller pre-heater may be used.

FIG. 1C is a schematic diagram of a second alternative embodiment of the PFC abatement system 100a of FIG. 1A, referred to as PFC abatement system 100c. The PFC abatement system 100c is similar to the PFC abatement system 100a of FIG. 1A, but may employ a fuel source to pre-heat gas before entry into the catalyst bed 120. A cross heat exchanger, recuperator and/or pre-heater also may be used.

The byproducts of hydrocarbon combustion are water vapor and CO2. Using a fuel source such as natural gas, LPG, methane or the like to heat the gas stream before the gas stream contacts the catalyst in the cataylst bed 120 may add hydrogen in the form of water vapor, and provide a lower cost of operation than the use of electricity for heating. The heating of the gas stream with the fuel also destroys some PFCs that are easier to abate by temperature alone, and/or leaves PFCs with lower numbers of carbon atoms (rendering the PFCs easier to destroy by catalysts).

With reference to FIG. 1C, the system 100c includes a fuel source 170 for adding a fuel such as natural gas to the gas stream to be abated, along with excess air (e.g., in a combustion region or chamber 172). The fuel/air mixture is ignited either with an electric spark, a hot surface ignitor such as a hot metal surface, or a standing pilot 174. Alternatively excess air may be added, and then fuel, possibly with a premix of some air, to insure a stable flame without the formation of soot.

### Exemplary System Components

Wet Scrubber

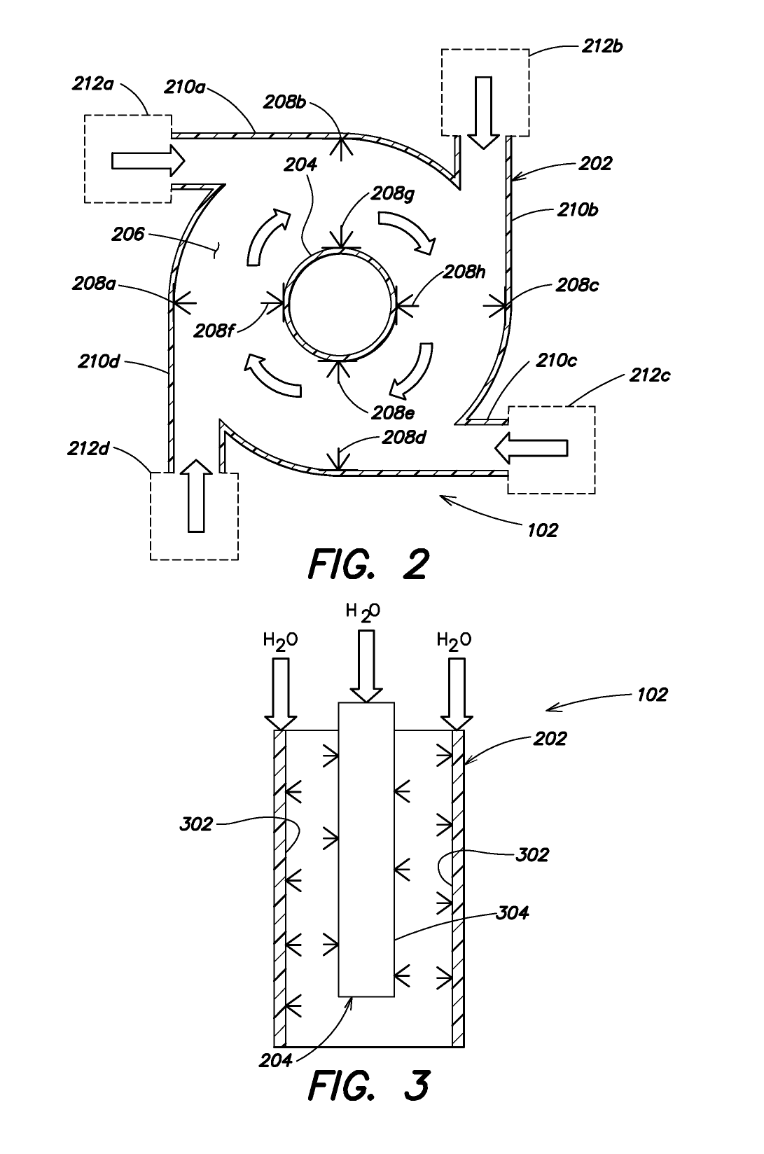

As stated above, the wet scrubber 102 is adapted to use a water mist to remove contaminants, such as SiF₄, from the gaseous waste stream(s) output by the process tool 106. For example, a plurality of high pressure nozzles may be used to create a mist within the wet scrubber 102. Exemplary embodiments of the wet scrubber 102 are described below with reference to FIGS. 2-4.

FIG. 2 is a top perspective view of an exemplary embodiment of the wet scrubber 102 depicted in FIGS. 1A-C; and FIG. 3 is a cross-sectional view of the wet scrubber of FIG. 2. In the embodiment of FIGS. 2-3, wet scrubber 102 includes a set of concentrically nested tubes (e.g., an outer tube 202 and an inner tube 204). The outer tube 202 and inner tube 204 define an inner cavity 206 through which gaseous waste streams from one or more process tools and/or process chambers may pass. Water and/or other gases and/or fluids may be directed through outer tube 202 and inner tube 204 and dispensed radially into the inner cavity 206 via spray nozzles 208a-h. Though depicted in FIG. 2 as four columns of nozzles spaced equally apart on both outer tube 202 and inner tube 204, it is understood that any number and/or arrangement of spray nozzles 208a-h may be utilized.

With reference to FIG. 2, the wet scrubber 102 includes four inlets/conduits 210a-d, each adapted to receive a gaseous waste stream from a process chamber 212a-d (shown in phantom). In general, the wet scrubber 102 may include any number of inlets/conduits, and each inlet/conduit may be coupled to one or more process chambers and/or process tools. Also, a single process chamber may be coupled to more than one of the inlets/conduits 210a-d.

In the embodiment of FIGS. 2-3, the inlet/conduits 210a-d are arranged so that each inlet/conduit 210a-d directs a gaseous waste stream approximately tangentially along a first inner surface 302 (and/or a second inner surface 304) of the water scrubber 102. Such an arrangement increases the residence time of gaseous waste streams within the wet scrubber 102 thereby increasing the effectiveness of any water scrubbing process performed therein. Other inlet/conduit configurations may be used.

Outer tube 202 and inner tube 204 may be constructed of plastic or other materials and may be lined with plastic and/or other materials to prevent deposition of particles in the gaseous waste streams. Inner cavity 206 may be sealable such that inlets to the inner cavity 206 are confined to spray nozzles 208a-h and inlet/conduits 210a-d and outlets from the inner cavity 206 are confined to one or more conduits 112 (FIGS. 1A-C). In at least one embodiment, outer tube 202 may be of a conical shape having a smaller diameter at its bottom. This shaping may promote efficient run-off of water and prevent particulates and other unwanted debris from accumulating within inner cavity 206. In an exemplary embodiment, the inner cavity 206 of wet scrubber 102 may have a volume of approximately 5-10 liters, although any larger or smaller sizes may be used.

Water and/or other gases and/or fluids may be directed through outer tube 202 and inner tube 204 and dispensed radially into the inner cavity 206 via spray nozzles 208a-h. Spray nozzles 208a-h may be atomizer type spray nozzles and may dispense a high pressure mist of water droplets. In some embodiments, spray nozzles 208a-h may dispense water droplets of a diameter of about 10 to 100 microns, and more preferably about 50 microns or less. Larger and/or smaller water droplet sizes may be dispensed. In at least one embodiment of the wet scrubber 102, atomizing water nozzles are employed to produce drops of about a 10 to 100 micron diameter so as to create an approximately 0.1 to 5 second, and preferably about 2.5 to 5 second, contact time between water particles and the gaseous waste stream(s). Spray nozzles 208a-h and/or other water dispensers may also direct a water curtain along the first and second inner surfaces 302 and 304 of the inner cavity 206 to prevent deposition of particulates on these surfaces.

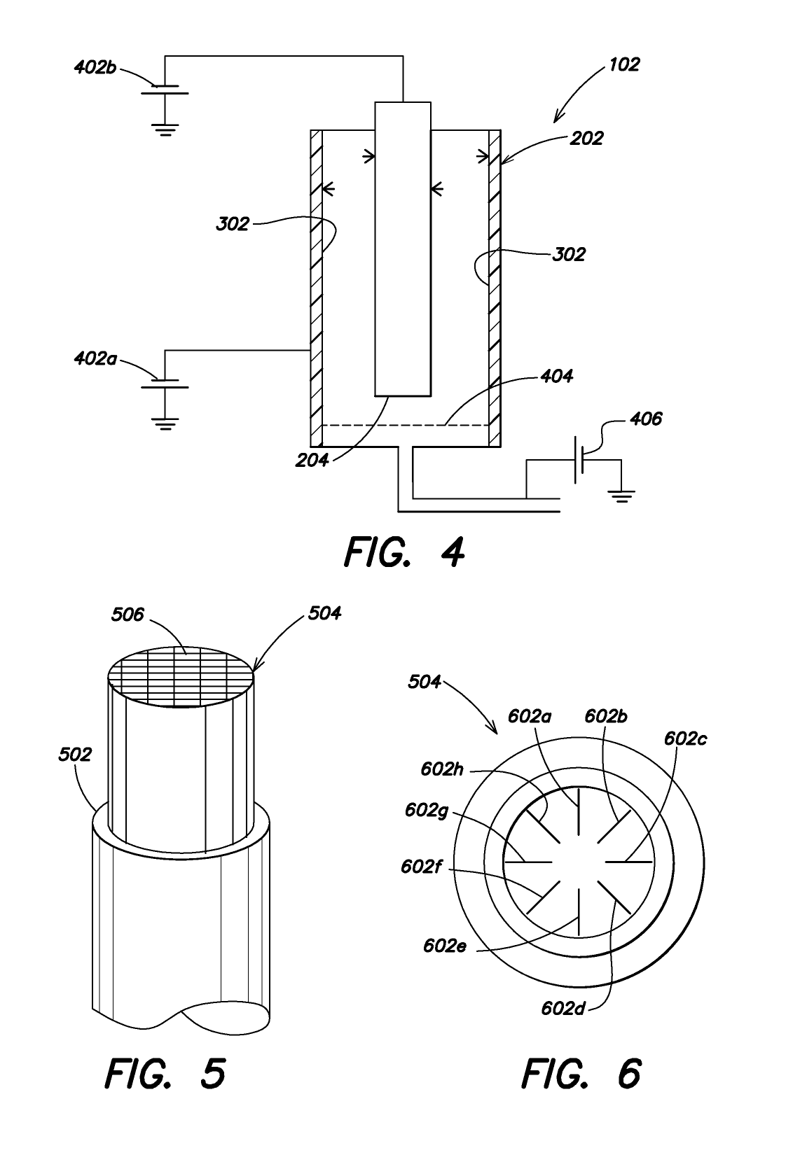

FIG. 4 is a cross-sectional view of an alternative embodiment of the wet scrubber 102 of FIG. 3. In the embodiment of FIG. 4, water droplets dispensed by spray nozzles 208a-h may be electrostatically enhanced. For example, biasing electrodes may charge water droplets dispensed by spray nozzles 208a-h to prevent the water droplets from coalescing. A positive or negative charge may be applied to water droplets by coupling a first charger 402a (e.g., a DC voltage supply) to the outer tube 202 and the same or a second charger 402b to the inner tube 204 of the wet scrubber 102. As all water droplets have the same charge, the droplets repel each other, preventing and/or minimizing coalescence. The voltage applied to the inner/outer tubes may range from about 100 to 5000 volts, although larger or smaller voltages may be used.

A metal or otherwise conducting grid 404 may be positioned near the bottom of wet scrubber 102 to collect the charged water droplets. For example, as water droplets fall onto the grid 404, the droplets will be collected by the grid and lose their charge, allowing the droplets to coalesce and fall through conduit 112 into sump 114. The grid 404 may be grounded, floating or charged to an opposite polarity relative to the droplets. The grid 404 may be constructed of wire mesh or any other suitable material. In some embodiments, the grid 404 may be additionally and/or alternatively positioned before and/or after first packed bed chamber 110. Other systems and/or methods to control water droplet size, direction of travel, and/or formation may be employed in wet scrubber 102. For example, in addition to or in place of the grid 404, a bottom or outlet of the wet scrubber 102 may be grounded, floating or charged (as indicated by reference numeral 406) to an opposite polarity relative to the droplets.

First Packed Bed Chamber

Referring again to FIGS. 1A-C, the first packed bed chamber 110 or “demister” removes any “fog” in gaseous waste streams received from the wet scrubber 102. In some embodiments, the first packed bed chamber 110 may include a packed bed of beads, barrels, or other shapes formed from ceramic, metal alloy, polypropylene, and/or any other suitable material. A plurality of nozzles 136 near an outlet of the first packed bed chamber 110 create a stream or rainfall of water that flows (via gravity) down the packed bed to the sump 114. In this manner, mist introduced to the gaseous waste stream(s) by the wet scrubber 102 is removed. The nozzles 136 may operate continuously or intermittently.

In some embodiments, the first packed bed chamber 110 may be a sealable tube arranged such that gaseous waste streams are directed via conduit 112 into a lower end of the packed bed chamber. As stated, the first packed bed chamber 110 may be packed (e.g., filled or partially filled) with material for trapping, removing, and/or abating liquid water, water vapor, chemicals, and/or particulates in gaseous waste streams. Exemplary packing materials may include polypropylene, metal alloys, polymers, alumina, ceramics, etc., that are barrel-shaped, ring-shaped, bead-shaped and/or otherwise shaped. Other shapes and/or materials may be used (e.g., such as for high temperature or corrosive applications) The first packed bed chamber 110 may, in some embodiments, have an interior volume of approximately between four and eight liters. Packed bed chambers of larger or smaller volumes may be employed, as appropriate.

Note that a gaseous waste stream may be flowed in a counter-current and/or optionally a co-current manner through the packing with and/or against the flow of water. Air may be injected to provide direct cooling and promote reduction of the humidity of the exiting gaseous waste stream.

Pressure Regulator

Blower 118 may be constructed of plastic or other corrosion resistant materials, and may be attached directly to the first packed bed chamber 110, the catalyst bed 120, or indirectly to either or both of these units via appropriate conduits (as shown in FIGS. 1A-C). The blower 118 may serve to apply positive pressure to the catalyst bed 120. In some embodiments, the pressure applied may be approximately five in. W.C., although more or less pressure may be applied as appropriate. In the same or alternative embodiments, the blower 118 may be controllable in real time to maintain an approximately constant pressure within the system 100, especially within the catalyst bed 120, as will be discussed below.

In an alternative embodiment, the blower 118 may be replaced by a passive device, such as an eductor or another pressure regulator. Use of such a passive device may reduce operating expenses. In such an embodiment, the eductor may take in a small amount of high pressure CDA (“Clean Dry Air”) that is mixed with the gaseous waste stream from the first packed bed chamber 110. This increases the flow rate of the gaseous waste stream sent to the catalyst bed 120.

In some embodiments, it may be desirable to track and/or control pressure and/or flow in the abatement system 100. For example, pressure in the abatement system 100 may be measured by one or more pressure indicators 138a-c. Pressure indicators 138a-c may measure pressure at the first packed bed chamber 110 outlet, the catalyst bed 120 inlet, and/or immediately before passing to house exhaust 130, respectively. These locations may be utilized to determine pressure in and/or pressure drop across the catalyst bed 120. Additional pressure indicators may be located wherever it is desirable to track and/or control pressure in the abatement system 100.

The pressure indicators 138a-c may detect clogging in the second packed bed chamber 122 and the catalyst bed 120. Also, the pressure indicators 138a-c may allow balancing of the pressure at the first packed bed chamber 110 outlet and the catalyst bed 120 outlet. This balancing may prevent water from being drawn from the sump 114 into the first packed bed chamber 110 and/or into the catalyst bed 120 should a large pressure differential be created across these components. Pressure indicators 138a-c may be any sensors capable of detecting pressure or differential pressure such as slant manometers, orifice plates, diaphragms, etc. Blower 118 may also be equipped with a damper and/or pressure switch to assist control of pressure within the abatement system 110.

Flow into blower 118 (or an eductor) may be controlled by a flow regulator 140. Flow regulator 140 may be any device capable of controlling gas and/or liquid flow such as a mass flow controller.

A controller 142 may be connected to and capable of receiving information from and/or transmitting command signals to blower 118, pressure indicators 138a-c, and/or flow regulator 140. For example, the controller 142 may adjust (e.g., in real time) the pressure in the abatement system 100, such as the pressure drop across catalyst bed 120. In some embodiments, the controller 142 may control the speed of the blower 118 to regulate pressure, or control the flow rate of CDA, compressed air, or other motive into an eductor to regulate pressure. Controller 142 may be a computer, microcontroller or any other appropriate hardware and/or software.

Catalyst Bed

The catalyst bed 120 may, in some embodiments, be formed from a conventional thermal oxidation and/or combustion chamber. For example, the catalyst bed 120 (and the second packed bed 122) may be a retrofitted CDO chamber 143, such as a retrofitted version of the EcoSys CDO863 manufactured by Metron Technology, Inc. of San Jose, Calif. Such a CDO chamber 143 is generally cylindrical and includes heaters 144 adapted to heat an inner cavity 146 of the chamber (defined by a liner 148) during thermal oxidation processes. In an exemplary embodiment, the catalyst bed 120 may have an interior volume of approximately 4.7 to 6.4 liters, although larger or smaller volumes may be used.

It will be understood that the abatement system 100 may use a catalyst bed 120 that is not formed from a retrofitted CDO chamber. However, use of existing, on-site abatement equipment such as a CDO chamber that is retrofitted to abate PFCs can result in a significant cost savings when compared to the expense of installing an entirely new PFC abatement system.

FIG. 5 is a partial perspective view of a CDO chamber 502 that may be used as the catalyst bed 120 in accordance with the present invention. The CDO chamber 502 may be a cylindrical, tubular or other shape. To allow the CDO chamber 502 to abate PFCs, the CDO chamber 502 is filled with a catalyst (e.g., as the CDO chamber 502 typically cannot be heated to a sufficient temperature to directly abate PFCs). In some embodiments, the interior of the CDO chamber 502 and/or catalyst bed 120 may be lined with and/or constructed of corrosion resistant metals or ceramics (e.g., Inconel™ or Hastelloy™, nickel, yttria doped alumina, titania with alumina, etc.) and/or other corrosion resistant materials with high thermal conductivity.

A catalyst may be directly placed into the CDO chamber 502 (filling or partially filling the CDO chamber 502). In an alternative embodiment, a removable and/or readily serviceable catalyst cartridge 504 that is prefilled with a catalyst may be inserted into the CDO chamber 502. The catalyst cartridge 504 may also be of a cylindrical or tubular shape, and in some embodiments capped on each end by screens 506 or other porous structures that allow gaseous waste streams to travel through the catalyst trapped by the screens 506.

As stated, the catalyst may aid in the reaction and/or destruction of components of gaseous waste streams by lowering the reaction temperature for the abatement of PFCs. Destruction of PFCs may require reaction temperatures in the range of approximately 950° C. to approximately 1300° C. Use of a catalyst may lower a reaction temperature for PFCs to approximately 500° C. in some embodiments.

Exemplary catalysts may include: ceramics; calcium magnesium; barium or strontium oxide; hydroxide; carbonate; nitrate; phosphates of aluminum, boron, alkali earth metal, titanium, zirconium, lanthanum, cerium, yttrium, rare earth metal, vanadium, niobium, chromium, manganese, iron, cobalt and/or nickel; metals of groups 4 to 14 of the periodic table; iron oxide; alumina; zirconia; titania; silica; vanadium oxide; tungsten oxide; tin oxide; platinum; palladium; rhodium; gamma alumina; cobalt oxide; and/or cerium. Other catalysts may be used. In one particular embodiment, inverse spinel crystal structure manganese may be used. Reaction catalysts may be formed or be of any appropriate shape (e.g., rings, beads, barrels, honeycomb, etc.).

FIG. 6 is a top view of an exemplary embodiment of the cartridge 504. With reference to FIG. 6, to control temperature of the catalyst and/or within the catalyst bed 120, the heaters 144 (FIGS. 1A-C) may be employed. The heaters 144 may be cylindrical so as to conform to the shape of outer chamber 502 and provide heat to the liner 148 and the catalyst bed 120. To allow more uniform heating across the catalyst bed, thermal fins 602a-h may be provided within the cartridge 504. The thermal fins 602a-h may be constructed of metal or another thermally conductive material, run the vertical length of the heaters 144, and/or may be arranged radially toward the center of catalyst bed 120. Heat thereby may be more uniformly transferred from the heaters 144 to the catalyst bed 120. Other numbers of thermal fins or other types of thermal conduction mechanisms may be employed. The cartridge 504 may be formed from the same or a different material than the thermal fins 602a-h.

In some embodiments, the catalyst bed 120 may be double contained by use of an outer shell (not shown) such that gaseous waste streams may not escape abatement system 100 at the catalyst bed 120. In the same or other embodiments, the catalyst bed 120 may have additional exhaust to remove some portion of a gaseous waste stream.

As another example, the catalytic bed 120 may include a catalytic surface that catalyzes a reaction for reducing the hazardous gas content in gaseous waste streams. For example, PFCs, as well as residual halogens (e.g., fluorine), HAPs and/or VOCs, may be abated via a reaction between a gaseous waste stream and a catalyst present in the catalytic bed 120.

The catalytic surface of the catalyst bed 120 may be, for example, a structure made from catalytic material or supporting a finely divided catalyst, a bed of foam or pellets, or a coating on a wall or component of the catalytic bed 120. For example, the catalytic surfaces may comprise surfaces of a support structure comprising a honeycomb member with the catalyst embedded therein to form a high surface area member over and through which the effluent passes as it flows from an inlet to an outlet of the catalyst bed 120. The catalytic surfaces may be on, for example, a structure comprising a ceramic material, such as cordierite, Al₂O₃, alumina-silica, alumina-titania, mullite, silicon carbide, silicon nitride, zeolite, and their equivalents; or may comprise a coating of materials, such as ZrO₂, Al₂O₃, TiO₂ or combinations of these and other oxides. The catalytic surfaces may also be impregnated with catalytic metals, such as Mn, Pt, Pd, Rh, Cu, Ni, Co, Ag, Mo, W, V, La or combinations thereof or other materials known to enhance catalytic activity.

In general, decreasing the size of the grains or other structure of the catalyst in the catalyst bed 120 may increase the surface area and effectiveness of the catalyst. However, such size reduction may also increase the pressure drop of gas flowing through the catalyst bed 120.

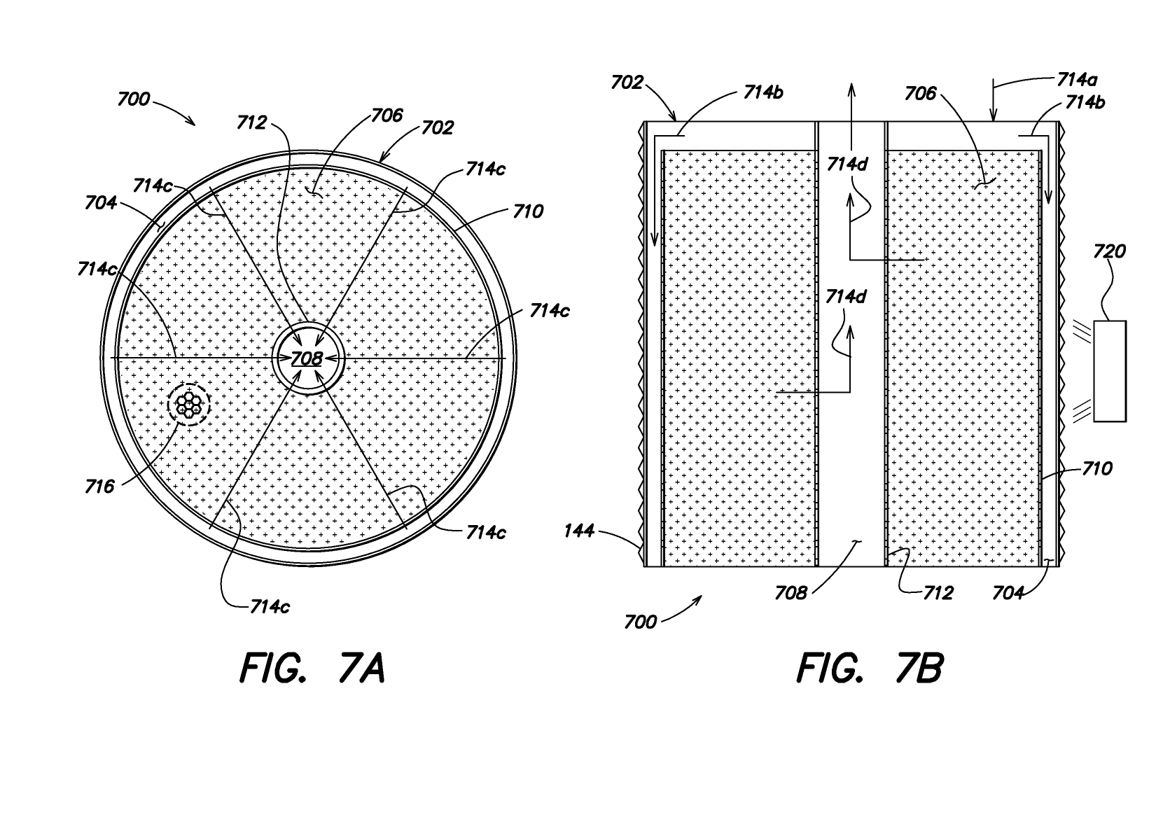

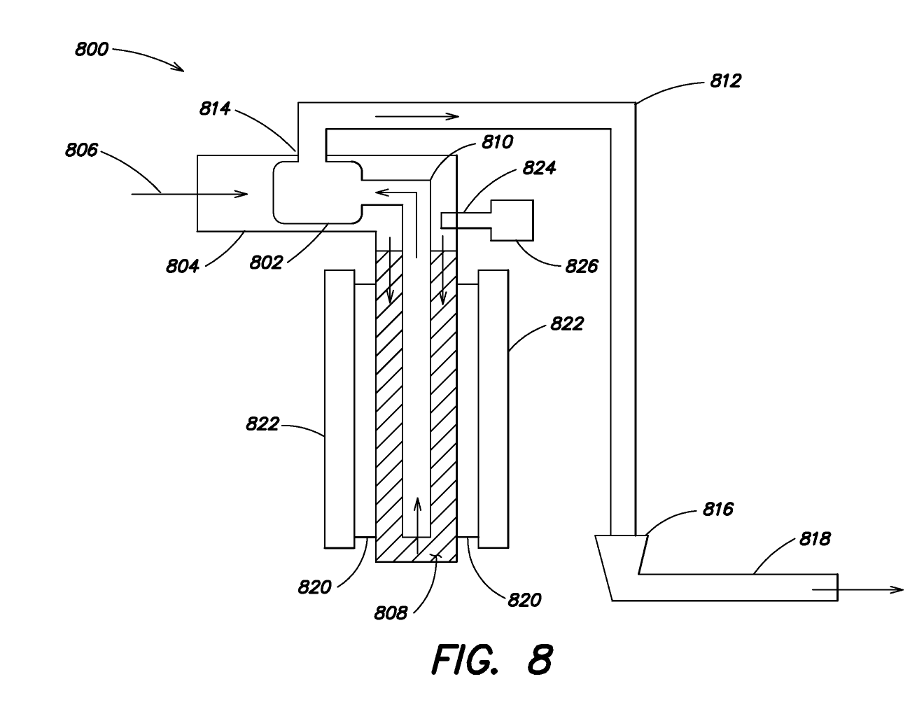

In some embodiments, a vacuum generator (not shown, such as a vacuum pump) may be employed at or near the end of the catalyst bed 120 to compensate for any pressure drop produced by the catalyst bed 120. In the same or other embodiments, pressure drop through the catalyst bed 120 may be reduced by the geometry of the catalyst bed 120. For example, FIGS. 7A and 7B are a top view and a side view, respectively, of an exemplary reduced-pressure-drop catalyst bed 700 provided in accordance with the present invention that may be used in any of the abatement systems described herein.

With reference to FIGS. 7A and 7B, the catalyst bed 700 includes a reactor chamber 702 having an annular plenum 704 along the length of the reactor chamber 702 outside of catalyst material 706 of the catalyst bed 700, and an inner plenum 708 that extends through a central region of the reactor chamber 702 and catalyst material 706.

The outer plenum 704 may be formed, for example, by positioning an outer porous liner 710 within the reactor chamber 702 and spaced from an inner surface of the reactor chamber 702 so as to define the outer plenum 704. The inner plenum 708 may be formed from an inner porous liner 712 positioned within a central region of the reactor chamber 702 so as to define the inner plenum 708. The outer and inner liners 710, 712 contain the catalyst material 706 within the reactor chamber 702. In the embodiment shown, the outer and inner liners 710, 712 may be formed from porous tubes, sheets or cylinders, such as porous ceramic, perforated metal, etc., tubes, sheets or cylinders. Other materials and/or shapes may be used.

In operation, a gaseous waste stream to be abated flows into the outer plenum 704 of the catalyst bed 700 (arrow 714a in FIG. 7B) and may flow freely along the length of the reactor chamber 702 (arrows 714b in FIG. 7B). Due to the porous nature of the outer liner 710, the gaseous waste stream travels radially through the outer liner 710 (arrows 714c in FIG. 7B), through the catalyst material 706, through the inner liner 712 and into the inner plenum 708 (arrows 714d in FIG. 7B) as shown. The gaseous waste stream then exits the catalyst bed 700.

The geometry of the catalyst bed 700 significantly enhances and/or maximizes the cross sectional area of catalyst material 706 that contacts the gaseous waste stream, while significantly reducing and/or minimizing pressure drop across the catalyst bed 700. It will be understood that gas flow direction may be reversed. For example, a gaseous waste stream may enter the catalyst bed 700 from the inner plenum 708 and travel through the inner liner 712, through the catalyst material 706, through the outer liner 710 and into the outer plenum 704 where it exits the catalyst bed 700.

In at least one embodiment, the reactivity of the catalyst in the catalyst bed 700 (or any other catalyst bed described herein) may be enhanced with electromagnetic radiation. For example, pulsed microwaves may be applied to a catalyst bed so as to cause a polarizability catastrophe to a catalyst surface that enhances catalytic reactivity. U.S. Pat. No. 6,190,507, which is hereby incorporated by reference herein in its entirety, describes the use of short burst, high-power microwave fields to increase the reactivity of the surface of a catalyst. In one embodiment, microsecond bursts of about 5 GHz microwaves with about 40 psec rise times may be employed.

Most catalytic PFC abatement systems utilize a granular or pellet form of catalyst or catalyst support. These pack tightly and typically exhibit high pressure drop.

In some embodiments of the invention, porous yttrium doped, zirconia stabilized alumina may be employed as a high surface area catalyst support to significantly reduce the pressure drop in the catalytic bed 120. Such as support is capable of withstanding a corrosive high temperature environment without breaking down. A catalytic support may be fabricated in various different shapes. For example, a support may be fabricated in cylinders, disks or other suitable shapes that fit within the inner cavity of the catalyst bed 120. The vertical dimension of the catalyst bed 120 may be filled by stacking these cylindrical or disk-shaped catalyst supports. If the catalytic bed 120 becomes plugged, the plugging generally is confined to the upper portions of the bed, and may be resolved by simply replacing only the top catalyst cylinders or disks as needed.

PFCs require high temperatures for complete destruction, especially CF4 which requires temperatures in excess of about 1100° C. These high temperatures may be difficult to achieve with electrically heated systems. Using catalysts specific for PFCs allows PFC destruction temperatures to be reduced, in some embodiments, to between about 500-800° C.

PFC catalysts typically require water, or a source of hydrogen and oxygen to keep from being deactivated. In some embodiments, the water may be provided by a pre-scrubber before the catalyst bed 120, such as by the wet scrubber 102 and/or the first packed bed chamber 110.

The gas stream may be heated before contacting the catalyst within the catayst bed 120, such as via a recuperator and/or heater as previously described with reference to FIG. 1B.

FIG. 8 illustrates a schematic view of a first apparatus 800 for heating a catalyst bed, such as the catalyst bed 120, 700 of FIGS. 1A-7B, provided in accordance with the present invention. With reference to FIG. 8, the first apparatus 800 includes a heat exchanger 802 inside of a reactor pipe 804 adapted to convey a waste stream (e.g., process by-products) entering in the direction shown by an arrow 806. The reactor pipe 804 may also have an abatement bed 808, such as a catalyst bed, in a portion of the reactor pipe 804. In this embodiment, the abatement bed 808 may be disposed about an inner pipe 810. As shown in FIG. 8, the inner pipe 810 may be coupled to the heat exchanger 802. The heat exchanger 802 may also be coupled to an exhaust pipe 812 through a wall of the reactor pipe 804 at an interface 814. The exhaust pipe 812 may be coupled to a quench 816. For example, the quench 816 may be the second packed bed chamber 122 of FIGS. 1A-C. The quench 816 may be coupled to a waste pipe 818 adapted to dispose of the treated waste stream (e.g., to the sump 114 of FIGS. 1A-C).

The first apparatus 800 may also include a reactor heater 820 and an insulator 822 disposed about the reactor pipe 804. As shown in FIG. 8, the reactor heater 820 and the insulator 822 are depicted in cross section views. A waste stream heater 824 may be disposed inside the reactor pipe 804. The waste stream heater 824 may be coupled to a power supply 826.

The heat exchanger 802 may be a coiled pipe of a steel alloy such as a Nickel-based alloy, for example Inconel 600 or 625™ available from Inco Corporation in Huntington, W. Va., although any suitable shape and/or material may be employed. For example, although a coil shape may be employed in the present embodiment, in the same or alternative embodiments a multi-fin shape may be used. Also, the material may be any suitable material adapted to carry a waste stream and transfer heat between a region inside the heat exchanger 802 and a region outside the heat exchanger 802. In some embodiments, the waste stream temperature may be about 800 to about 900 degrees Celsius although higher or lower temperatures may be present.

Similarly, the reactor pipe 804, the inner pipe 810, the exhaust pipe 812, and the waste pipe 818 may be formed from Inconel 600 or 625™, although any suitable material may be used. For example, in some embodiments a less expensive stainless steel alloy may be employed in the exhaust pipe 812 when the properties (e.g., corrosiveness, temperature, etc.) of the waste stream are not detrimental to the stainless steel. Although the reactor pipe 804, the inner pipe 810, the exhaust pipe 812, and the waste pipe 818 may be round pipes, in general, any suitable shape and/or sizes may be employed.

The temperature of the waste stream carried by the reactor pipe 804, the inner pipe 810, the exhaust pipe 812, and the waste pipe 818 may range from about room temperature to about 900 degrees Celsius although higher or lower temperatures may be present.

The reactor heater 820 may be a ceramic heater from, for example, the ceramic heater product line available from Watlow Corporation in St. Louis, Mo., although any suitable heater may be employed. The ceramic portion of the reactor heater 820 may provide some insulation. To provide additional insulation, the insulator 822 or any suitable insulation may be provided. The insulator 822 may also prevent injuries to operators and/or damage to equipment. As shown in FIG. 8, the insulator 822 may be wrapped around the reactor heater 820 although any suitable configuration of the reactor heater 820 and the insulator 822 may be employed to heat the reactor pipe 804 and the waste stream.

The waste stream heater 824 may be an electric heating device although any suitable heating device may be employed. As shown in FIG. 8, the waste stream heater 824 may have a portion inside the reactor pipe 804 so as to contact the waste stream inside the reactor pipe 804. Although FIG. 8 depicts the waste stream heater 824 as a rod, other configurations may be employed in the same or alternative embodiments. The waste stream heater 824 may be at a temperature that is higher than the temperature of the waste stream. Accordingly, the waste stream heater 824 may heat the waste stream around the waste stream heater 824 to a desired temperature. The waste stream heater 824 may heat the waste stream by using electricity supplied by the power supply 826 although any suitable power source may be employed.

In operation, the waste stream may enter the reactor pipe 804 as depicted by the arrow 806, and flow about the outer surface of the heat exchanger 802. As will be explained below, the heat exchanger 802 may be at a temperature that is greater than the temperature of the waste stream. Accordingly, heat is transferred from the heat exchanger 802 to the waste stream to heat the waste stream. The waste stream may flow past the heat exchanger 802 and the waste stream heater 824. The waste stream heater 824 may be at a temperature higher than the heat exchanger 802 although any suitable temperature may be employed. The waste stream heater 824 may heat the waste stream to a desired temperature (e.g., for abatement). Subsequently, the waste stream may filter through the abatement bed 808 (e.g., catalyst bed 120, 700 of FIGS. 1A-7B). During this filtering the waste stream may react (e.g., chemically, physically, etc.) with the abatement bed 808 so as to change the chemical composition of the waste stream to a more desirable chemical composition. The reaction may occur at an elevated temperature.

Note that, as shown in FIG. 8, the waste stream is heated by the heat exchanger 802 prior to being heated by the waste stream heater 824. Accordingly, the heat exchanger 802 may use the heat retained in the waste stream after the reaction with the abatement bed 808 to preheat the incoming waste stream.

After filtering through the abatement bed 808, the waste stream may flow through the inner pipe 810 into the heat exchanger 802. Because the waste stream may cool during the filtering, it may be at a temperature that is slightly less than the abatement temperature. However, the temperature of the waste stream after abatement is generally higher than the temperature of the entering waste stream. Accordingly, as discussed above, the heat exchanger 802 may heat the incoming waste stream. The abated waste stream may flow through the heat exchanger 802 and the exhaust pipe 812 towards the quench 816 (e.g., second packed bed chamber 122 of FIGS. 1A-C). The quench 816 may further cool and/or abate chemistries in the waste stream. Subsequently, the waste pipe 818 may dispose of the waste stream (e.g., to the sump 114 of FIGS. 1A-C).

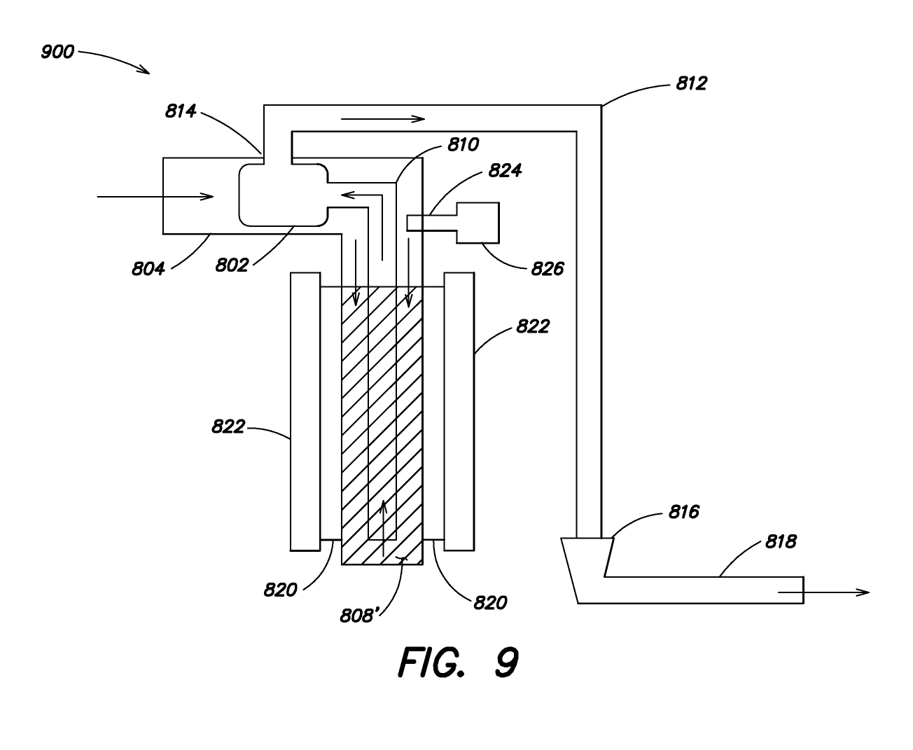

FIG. 9 illustrates a schematic view of a second apparatus 900 for heating a catalyst bed, such as the catalyst bed 120, 700 of FIGS. 1A-7B, provided in accordance with the present invention. With reference to FIG. 9, the second apparatus 900 may include an abatement bed 808′ (e.g., a catalyst bed) that may be similar to the abatement bed 808 of the first apparatus 800. As shown in FIG. 9, the second abatement bed 808′ is present inside the inner pipe 810.

In operation, the waste stream may flow as described above with reference to FIG. 8. The waste stream flows through the second abatement bed 808′ along a path that is longer than as described with reference to FIG. 8. Accordingly, the waste stream may have greater reaction and/or residence times with the second abatement bed 808′. Accordingly, the chemical composition of the waste stream may be abated more extensively.

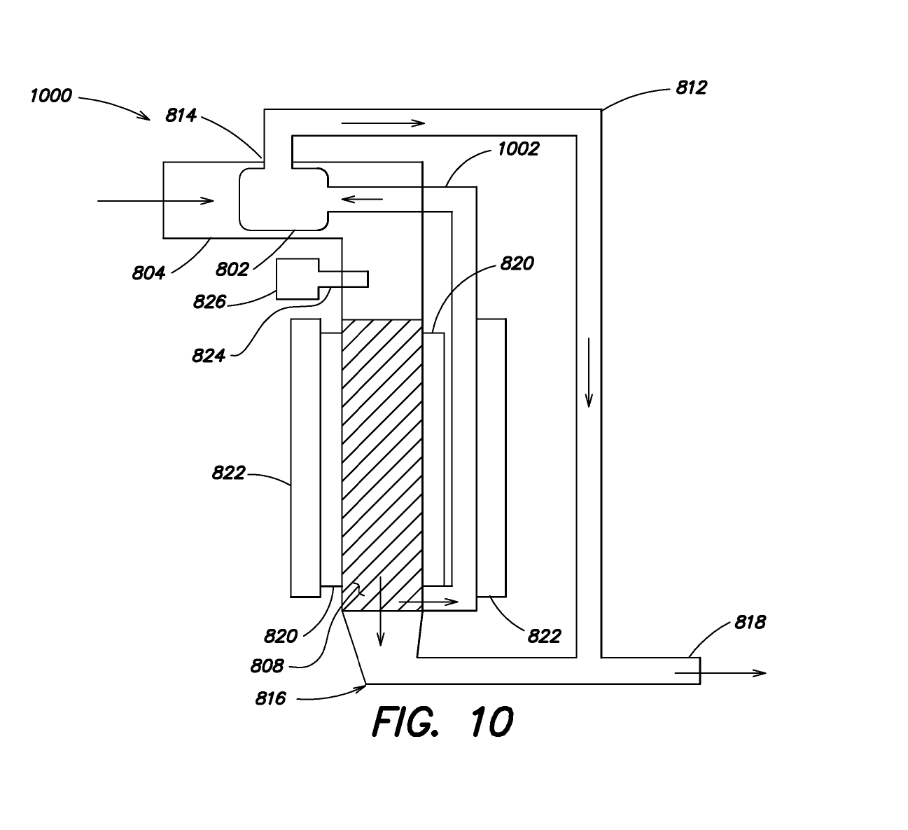

FIG. 10 illustrates a schematic view of a third apparatus 1000 for heating a catalyst bed, such as the catalyst bed 120, 700 of FIGS. 1A-7B, provided in accordance with the present invention. With reference to FIG. 10, the third apparatus 1000 may include an external pipe 1002 coupled to the reactor pipe 804 and the heat exchanger 802. The third apparatus 1000 may also include some components of the second apparatus 900. Note that the quench 816 is coupled to the reactor pipe 804. As shown in FIG. 10, a portion of the external pipe 1002 may be disposed outside the reactor pipe 804 and between the insulator 822 and the reactor heater 820 although any suitable configuration may be employed. For example, in alternative embodiments, the external pipe 1002 may be disposed between the reactor heater 820 and the reactor pipe 804. The external pipe 1002 may be similar to the inner pipe 810 described above with reference to FIG. 8. For example, the external pipe 1002 may be made of a nickel-alloy such as Inconel™ or another suitable material.

In operation, a waste stream may travel through the reactor pipe 804, through the abatement bed 808 and enter the external pipe 1002 at an elevated temperature. The abated waste stream may be conveyed by the external pipe 1002 between the reactor heater 820 and the insulator 822, thereby heating or preserving the temperature of the waste stream in the external pipe 1002. Subsequently, similar to the first apparatus 800 and the second apparatus 900, the abated waste stream may flow into the heat exchanger 802 to heat the heat exchanger 802 to a temperature higher than the temperature of the incoming waste stream. Accordingly, the heat exchanger 802 may preheat the incoming waste stream as described above with reference to FIGS. 8 and 9.

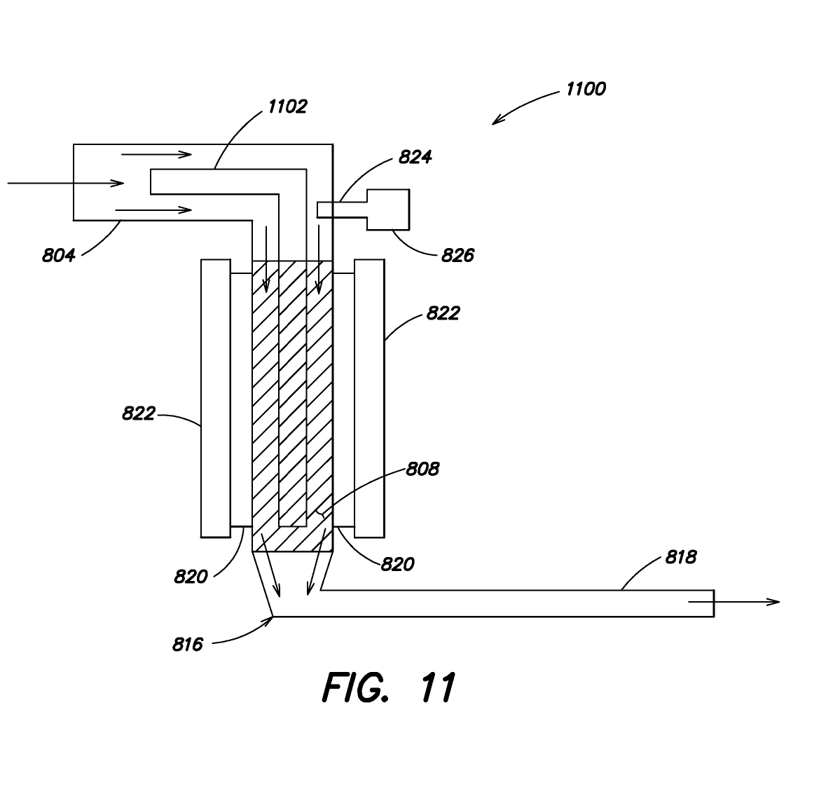

FIG. 11 illustrates a schematic view of a fourth apparatus 1100 for heating a catalyst bed, such as the catalyst bed 120, 700 of FIGS. 1A-7B, provided in accordance with the present invention. With reference to FIG. 11, the fourth apparatus 1100 may include a pipe 1102 in addition to some of the components described above with reference to FIG. 8. The pipe 1102 may be disposed in the abatement bed 808 inside the reactor pipe 804. As shown in FIG. 11, the pipe 1102 is disposed approximately center in the abatement bed 808 although any suitable location may be employed. A portion of the pipe 1102 extends beyond the abatement bed 808 into a region of the reactor pipe 804 in proximity to where the waste stream enters the reactor pipe 804.

The pipe 1102 may be a heat pipe although any suitable device may be employed. For example, the pipe 1102 may be a hollow heat pipe with a heat pipe fluid disposed inside the heat pipe. The heat pipe fluid may include a working fluid such as reduced pressure water, acetone, solvents, ammonia, etc., although any suitable fluid may be employed. The pipe 1102 may be similar to the material of the inner pipe 810 described above with reference to FIG. 8 although any suitable material may be employed. In FIG. 11, the pipe 1102 is a cylinder, although any suitable shape may be employed.

In operation, a first region of the pipe 1102 in the reactor heater 820 may increase to an abatement temperature (e.g., a temperature of the waste stream within the abatement bed 808, which may be, for example, a catalyst bed). Consequently, the heat pipe fluid may raise in temperature throughout the heat pipe 1102. For example, a portion of the heat pipe fluid may become gaseous and rise to a second region in proximity to where an incoming waste stream enters the reactor pipe 804. Because the heat pipe fluid is at a temperature greater than the temperature of the incoming waste stream, the heat pipe may transfer heat to the waste stream. The temperature of the incoming waste stream may increase, and the heat pipe fluid may condense back to a liquid form and flow back to the first region.

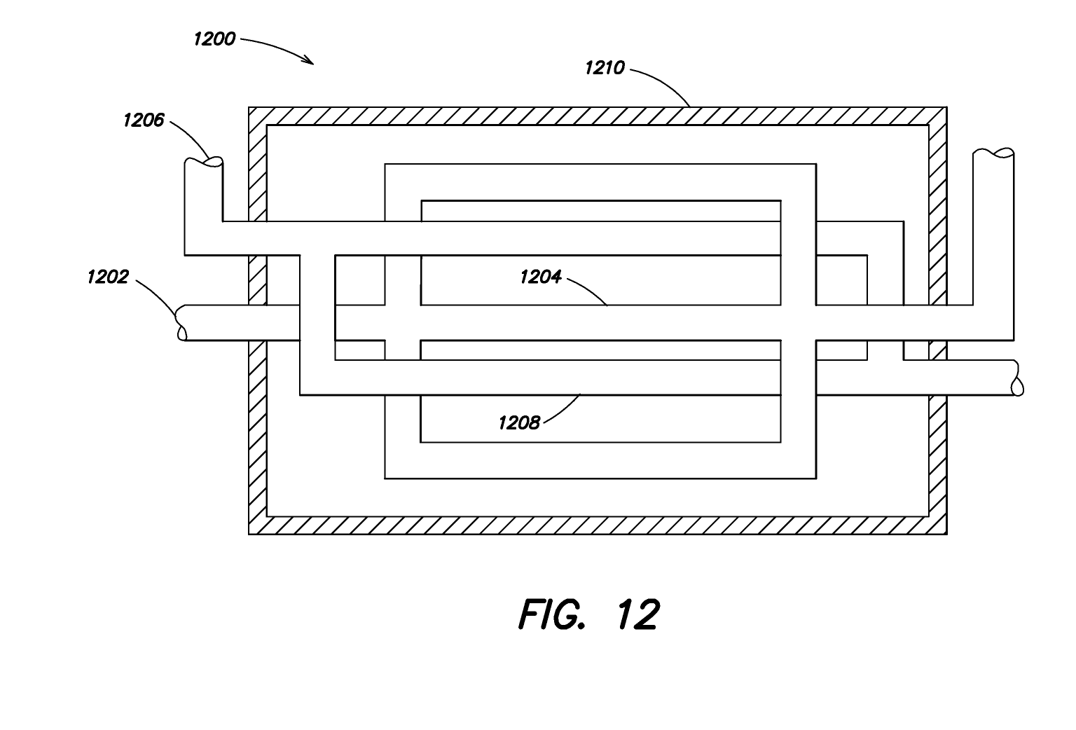

FIG. 12 is a schematic diagram of an exemplary cross heat exchanger 1200 that may be used for the heat exchanger 160 of FIG. 1B. Such a heat exchanger is similar to those described in previously incorporated U.S. Pat. No. 6,824,748.

With reference to FIG. 12, a gaseous waste stream to be abated (e.g., within the catalytic bed 120 of FIG. 1B) enters the cross heat exchanger 1200 at a first inlet 1202, and is dispersed into a first set of multiple channels 1204. An abated gas stream (e.g., catalytic bed 120) enters the heat exchanger at a second inlet 1206 and is dispersed into a second set of multiple channels 1208 which are adjacent and capable of transferring heat to the first multiple channels 1204 that carry the gaseous waste stream to be abated. Heat from the abated gas stream thereby is transferred to the gaseous waste stream to be abated. An insulating material 1210 may surround the heat exchanger 1200 to prevent the loss of heat to the atmosphere and to increase the efficiency of the heat exchanger 1200. The heat exchanger 1200 may be made of a corrosion resistant material such as a nickel-based alloy (e.g., Inconel®), or another suitable material.

Other types and/or number of heat exchangers may be used. For example, concentric tube heat exchangers in which hot gas flows within an inner tube and cold gas flows within an outer tube (or vice versa) may be employed, as may gas-to-gas heat exchangers.

Second Packed Bed Chamber

In some embodiments, the second packed bed chamber 122 may be of similar design and/or construction to the first packed bed chamber 110, discussed above. In at least one embodiment, the second packed bed chamber 122 may be part of the EcoSys CDO863 manufactured by Metron Technology, Inc. of San Jose, Calif. Other packed bed chambers may be used.

Referring again to FIGS. 1A-C, the second packed bed chamber 122 primarily removes acids and/or other undesirable by products of the PFC abatement that occurs in the catalyst bed 120. In some embodiments, the second packed bed chamber 122 may include a packed bed of beads, barrels and/or other shapes (not shown) formed from a corrosion resistant material such as ceramic or any other suitable material. A plurality of nozzles 150 near an outlet of the second packed bed chamber 122 create a stream or rainfall of water that flows (via gravity) down the packed bed to the sump 114. In this manner, acids (e.g., HF) and/or other components introduced to the gaseous waste stream(s) by the catalyst bed 120 are removed. The nozzles 150 may operate continuously or intermittently.

### Exemplary System Operation

In operation, gaseous waste streams from one or more process chambers (e.g., metal and/or dielectric etch chambers) are exhausted via exhaust lines 108a-d to wet scrubber 102. Water passed through high pressure pump 104 is atomized (e.g., pressed into droplets approximately 50 microns in size) and/or electrically charged at spray nozzles 208a-h. The gaseous waste streams are swirled around inner cavity 206 of wet scrubber 102 and through the fog of electrically charged water droplets, which react with the gaseous waste streams to remove and suspend in-water pollutants (e.g., SiF₄) that may harm downstream abatement equipment. Tangential insertion of the gaseous waste streams, as shown in FIG. 2, increases the residence time of the gaseous waste streams in wet scrubber 102. In an exemplary embodiment, the gaseous waste streams have a minimum residence time of at least approximately 0.1 seconds. Preferably, the residence time is approximately 2.5-5 seconds or more. Other residence times may be used as appropriate.

As water droplets contact grid 404, the water, SiF₄, and any other materials suspended in the water may flow out of water scrubber 102, through conduit 112 and branch 116 to sump 114. The unaffected portions of the gaseous waste streams may also flow through conduit 112 and then upward into the first packed bed chamber 110.

The first packed bed chamber 110 removes water (mist), contaminants, and/or particulates from the gaseous waste streams. The separated water, contaminants, and/or particulates may be directed to the sump 114 as described above. After passing through the first packed bed chamber 110, the gaseous waste streams may be directed to the blower 118 or an eductor (not shown). At this location within the abatement system 100, the gaseous waste streams primarily comprise a mixture of PFCs, nitrogen, non-soluble gases, and water vapor with acids, readily soluble by-products, particles, etc., from the process tool 106 removed.

When an eductor is employed in place of the blower 118, CDA, compressed air or another suitable gas may be added to the gaseous waste streams to affect pressure on the catalyst bed 120, and/or enhance, improve the efficiency of, and/or enable a reaction within the catalyst bed 120. When the blower 118 is employed, blower speed may be adjusted to achieve these objectives.

In the catalyst bed 120, the gaseous waste streams may be combusted, thermally oxidized, and/or otherwise reacted to abate PFCs (e.g., by converting PFCs to HF or other scrubbable by-products). After passing through the catalyst bed 120, the reacted gaseous waste streams are passed into conduit 124 through spray nozzles 126 to remove particulates and other contaminants generated by the catalyst bed 120. Particulates and other contaminants removed from the gaseous waste streams by spray nozzle water may be flowed with the water into the sump 114 via conduit 124 and branch 128.

The remaining gaseous waste streams may be flowed upwardly through the second packed bed chamber 122. Acids and/or particulates and contaminants thereby may be removed from the gaseous waste streams using the second packed bed chamber 122. Water from the sump 114 may be recirculated into the second packed bed scrubber 122.

Though not explicitly diagrammed in FIG. 1, it is understood that water that flows to high pressure pump 104 may also be flowed directly to first packed bed chamber 110, catalyst bed 120, water sprayers 126, the second packed bed chamber 122 and/or any water inlet and/or sprayer in the abatement system 100. Similarly water from sump 114, in some embodiments, may be recirculated to any desired location such as to the wet scrubber 102, the first packed bed chamber 110, the water sprayers 126, the second packed bed chamber 122, etc.

Gaseous waste streams may be passed to the house exhaust 130 for further abatement or exhaust after processing in the second packed bed chamber 122.

The foregoing description discloses only exemplary embodiments of the invention. Modifications of the above disclosed apparatus and method which fall within the scope of the invention will be readily apparent to those of ordinary skill in the art. For instance, to enhance PFC abatement, gaseous waste streams may be pre-heated before entering the catalyst bed 120. For example, hot nitrogen may be introduced to the gaseous waste streams near the inlet of the catalyst bed 120. Oxygen, air or enriched oxygen similarly may be injected into the gaseous waste streams near the inlet of the catalyst bed 120 to enhance abatement.

As stated, an eductor or air amplifier may be used in place of the blower 118. Additionally or alternatively, a blower, eductor or air amplifier may be used at the output of the second packed bed chamber 122 to affect, control and/or regulate pressure within the abatement system 100.

In some embodiments, the abatement system 100a-c may be used to abate hazardous air pollutants (HAPs) and/or volatile organic compounds (VOCs). The abatement system 100a-c may also include means for controlling pH at a desired location, such as near the recirculation pump 134 (e.g., using a port (not shown) for caustic injection).

Any number of scrubbers may be used before and/or after the catalyst bed 120 (e.g., 1, 2, 3, 4, etc.). Other types and/or number of heat exchangers may be used. For example, concentric tube heat exchangers in which hot gas flows within an inner tube and cold gas flows within an outer tube (or vice versa) may be employed, as may gas-to-gas heat exchangers.

In some embodiments, the catalytic bed 120 may be insulated and/or water-tight. The scrubbers may be co-current, counter-current, or a combination of the same. Other configurations may be used. An additional water heat exchanger may be used (e.g., for cooling recirculated water from the scrubbers).

In some embodiments, a blower or eductor (described above) may be positioned after the catalyst bed 120 and/or after the second packed bed chamber 122.

In some embodiments, a vacuum source, pump, or other vacuum generator 123 (FIG. 1C) may be employed at or near the end of the catalyst bed 120 to compensate for any pressure drop produced by the catalyst bed 120.

Any of the catalysts described herein may be formed or be of any appropriate shape (e.g., rings, beads, barrels, honeycomb as indicated, for example, by reference numeral 716 in FIG. 7A, or the like).

The catalytic surface of the catalyst bed 120 may be, for example, a structure made from catalytic material or supporting a finely divided catalyst, a bed of foam or pellets, or a coating on a wall or component of the catalytic bed 120. For example, the catalytic surfaces may comprise surfaces of a support structure comprising a honeycomb member (e.g., as indicated, for example, by reference numeral 716 in FIG. 7A) with the catalyst embedded therein to form a high surface area member over and through which the effluent passes as it flows from an inlet to an outlet of the catalyst bed 120.

In at least one embodiment, the reactivity of the catalyst in the catalyst bed 700 (or any other catalyst bed described herein) may be enhanced with electromagnetic radiation (e.g., from an electromagnetic radiation source 720). Note that any suitable location for a radiation source may be used.

Accordingly, while the present invention has been disclosed in connection with exemplary embodiments thereof, it should be understood that other embodiments may fall within the spirit and scope of the invention, as defined by the following claims.

The invention claimed is:

1. A method of abating perfluorocarbons (PFCs) in a gaseous waste abatement system having a controlled decomposition oxidation (CDO) thermal reaction chamber, comprising:
providing a catalyst bed within the CDO thermal reaction chamber;
conveying a gaseous waste stream past a heat exchanger into an inlet of the CDO thermal reaction chamber and to the catalyst bed;
filtering the gaseous waste stream through the catalyst bed, the filtered gaseous waste stream being heated in the catalyst bed; and
recirculating the heated gaseous waste stream from the catalyst bed to the heat exchanger.

2. The method of claim 1, further comprising:
introducing the gaseous waste stream along a first path into the CDO thermal reaction chamber; and
recirculating a portion of the heated gaseous waste stream downstream from the thermal reaction chamber via a second path having a portion running proximate to the first path;
wherein heat energy is transferred from the recirculated gaseous waste stream conveyed along the second path to the gaseous waste stream conveyed along the first path.

3. The method of claim 1, wherein the catalyst bed surrounds an inner conduit, the gaseous waste stream is filtered through the catalyst bed into the inner conduit so as to heat the gaseous waste stream, and the heated gaseous waste stream is recirculated through the inner conduit to the heat exchanger.

4. The method of claim 3, wherein the inner conduit includes catalyst material.

5. The method of claim 1, wherein the catalyst bed is coupled at a first end to an inlet of the thermal reaction chamber and coupled at a second end to an exit conduit, the gaseous waste stream is filtered through the catalyst bed and into the exit conduit so as to heat the gaseous waste stream and the heated gaseous waste stream is recirculated through the exit conduit to the heat exchanger.

6. The method of claim 1, further comprising:
re-heating the gaseous waste stream after the gaseous waste stream passes the heat exchanger and before the gaseous waste stream enters the catalyst bed.

7. A controlled decomposition oxidation (CDO) system for abating perfluorocarbons (PFCs) comprising:
an upstream portion including a first conduit adapted to convey a gaseous waste stream;
a thermal reaction chamber having an inlet coupled to the first conduit, a catalyst bed adapted to abate PFCs, and an outlet; and
a downstream portion including a second conduit having a first end coupled to the outlet of the thermal reaction chamber and having a portion, downstream from the first end, positioned proximate to the first conduit;
wherein the second conduit is adapted to convey a gaseous waste stream heated within the thermal reaction chamber to enable a transfer of heat energy from the second conduit to the first conduit so as to pre-heat the gaseous waste stream in the first conduit.

8. The controlled decomposition oxidation (CDO) system of claim 7, further comprising:
a heater positioned in the upstream portion so as to heat the gaseous waste stream in the first conduit after the gaseous waste stream has been pre-heated by transfer of heat from the second conduit.

9. A system for abating perfluorocarbons (PFCs) comprising:
an upstream portion including a first conduit adapted to convey a gaseous waste stream and a heating device coupled to the first conduit and adapted to pre-heat the gaseous waste stream; and
a thermal reaction chamber including an inlet coupled to the first conduit and a catalyst bed adapted to abate PFCs in the gaseous waste stream entering the thermal reaction chamber from the first conduit.

10. The system of claim 9, wherein the heating device comprises:
a chamber through which the gaseous waste stream is conveyed coupled to and adapted to receive combustible fuel supplied from a combustible fuel source; and
a pilot device positioned within the chamber adapted to ignite combustible fuel provided to the chamber.

11. A system for abating perfluorocarbons (PFCs) within a gaseous waste stream comprising:
a first conduit adapted to convey the gaseous waste stream and having an outlet;
a heat exchanger positioned in the first conduit proximate to the outlet;
a thermal reaction chamber including an inlet coupled to the outlet of the first conduit, a catalyst bed having a catalyst material positioned within the thermal reaction chamber adapted to abate PFCs within the gaseous waste stream; and
a second conduit having a first end coupled to the catalyst bed and a second end coupled to the heat exchanger.

12. The system of claim 11, wherein the heat exchanger comprises a coiled pipe composed of a steel alloy.

13. The system of claim 11, further comprising:
a reactor heater surrounding the thermal reaction chamber; and
an insulator surrounding the reactor heater.

14. The system of claim 11, further comprising:
a heating device positioned at the outlet of the first conduit adapted to re-heat the gaseous waste stream after the gaseous waste stream passes through the heat exchanger and before the gaseous waste stream is exposed to the catalyst bed.

15. The system of claim 14, wherein the heating device comprises an electric heater.

16. The system of claim 15, wherein the second conduit is substantially embedded within the catalyst bed and the first end of the second conduit is positioned in the catalyst bed.

17. The system of claim 16, wherein a portion of the second conduit is filled with catalyst material.

18. The system of claim 14, wherein the thermal reaction chamber includes an outlet positioned opposite from the inlet and the first end of the second conduit is positioned proximate to the outlet of the thermal reaction chamber.

19. A system for abating perfluorocarbons (PFCs) within a gaseous waste stream comprising:
a first conduit adapted to convey the gaseous waste stream and having an outlet;
a thermal reaction chamber including an inlet coupled to the outlet of the first conduit, a catalyst bed having a catalyst material positioned within the chamber and adapted to abate PFCs within the gaseous waste stream, and an outlet positioned opposite the inlet; and
a second conduit having a first end coupled to the catalyst bed and a second end that extends into the first conduit.

20. The system of claim 19, wherein the second conduit is substantially embedded within the catalyst bed and the first end of the second conduit is positioned in the catalyst bed.

21. The system of claim 19, wherein the second conduit includes a working fluid.

22. The system of claim 21, wherein a portion of the second conduit is filled with catalyst material.

23. The system of claim 21, further comprising:
a reactor heater surrounding the thermal reaction chamber;
an insulator surrounding the reactor heater; and
a heating device positioned at the outlet of the first conduit adapted to re-heat the gaseous waste stream after the gaseous waste stream passes by the second end of the second conduit and before the gaseous waste stream enters the catalyst bed.

24. The system of claim 19, further comprising:
a quench coupled to the outlet of the thermal reaction chamber.
